(19) United States
(12) Patent Application Publication
(10) Pub. No.: US 2014/0147855 A1
(43) Pub. Date: May 29, 2014

(54) Method for Extracting RNA from Fixed Embedded Tissue

(71) Applicants: Johannes Kratz, Cambridge, MA (US); David Berryman, Foster City, CA (US)

(72) Inventors: Johannes Kratz, Cambridge, MA (US); David Berryman, Foster City, CA (US)

(73) Assignee: PINPOINT GENOMICS, INC., Carlsbad, CA (US)

(21) Appl. No.: 14/233,368

(22) PCT Filed: Jun. 29, 2012
(86) PCT No.: PCT/US2012/045136

§ 371 Date: Jan. 16, 2014

Related U.S. Application Data
(60) Provisional application No. 61/504,133, filed on Jul. 1, 2011.
(60) Provisional application No. 61/504,202, filed on Jul. 2, 2011.

(51) Int. Cl. C12Q 1/68

(52) U.S. Cl. CPC C12Q 1/6806
USPC 435/6.12

(57) ABSTRACT

The present invention provides methods for extracting RNA from formalin fixed embedded tissue wherein the extracted RNA provides reliable results in assays for RNA quantity such as PCR assays.

### CROSS-REFERENCE TO RELATED APPLICATIONS

This application claims the benefit of U.S. Provisional Patent Application Ser. No. 61/504,133, filed Jul. 1, 2011, and U.S. Provisional Patent Application Ser. No. 61/504,202, filed Jul. 2, 2011, both of which are incorporated herein by reference in their entirety.

### BACKGROUND OF THE INVENTION

Formalin-fixed, embedded tissue is the most widely available material for retrospective clinical studies. Analysis of gene expression and correlation with clinical parameters has the potential to become an important factor in therapeutic decision making. In addition, the ability to analyze gene expression in these tissues may make these tissues an invaluable resource for the elucidation of disease mechanisms and validation of differentially expressed genes as novel therapeutic targets and/or prognostic indicators. However, an obstacle to this approach has been the quantity and quality of RNA extracted from these tissue samples.

### BRIEF SUMMARY OF THE INVENTION

Methods for the extraction of total RNA from formalin fixed embedded tissue of sufficient quality and quantity to be measured by Polymerase Chain Reaction technologies are described herein. Generally, the methods include: the removal of the embedding material from the sample; incubation of the tissue with a protease to dissolve protein cross-links and release RNA from the protein-nucleic acid matrix; removal of protein and precipitation of the nucleic acids; removal of contaminating DNA; and purification of RNA using silica gel membranes.

In some embodiments the tissue sample is formalin fixed paraffin embedded (FFPE) tissue.

In some embodiments the paraffin is removed by xylene and alcohol washes. In preferred embodiments the alcohol is ethanol.

In some embodiments the protease to dissolve the protein cross-links has proteinase K activity, and in preferred embodiments is proteinase K.

In some embodiments the precipitation of nucleic acids is with alcohol containing solution, and in preferred embodiments the alcohol in the solution is isopropanol.

In some embodiments removal of the DNA is by digestion with DNase that is an endonuclease, and in preferred embodiments the DNase is DNase I.

Purification of RNA from the above treated samples is by precipitation on and elution from silica gel membranes. The silica gel membranes have the RNA, DNA, salt and protein binding characteristics of those in Qiagen RNeasy MinElute® Spin Columns of those in Qiagen RNeasy MinElute® Spin Columns, and in preferred embodiments the purification is by use of Qiagen RNeasy MinElute® Spin Columns

### BRIEF DESCRIPTION OF THE DRAWINGS

FIG. 1A is a graph showing the Ct values of RNA extracted from FFPE tissue using Methods 1 (Silica gel method) and Method 3 (PPG method); FIG. 1B is a graph showing the Ct values of RNA extracted from FFPE tissue using Method 1 (silica-membrane based method) and Method 2 (alcohol precipitation based method).

FIG. 2 is a trace of Nanodrop spectrophotometry of PPFE samples extracted by the PPG method and of RNA extracted from frozen tissue. The trace in black is of the RNA extracted from frozen tissue.

FIG. 3 is a graph showing prognosis for lung adenocarcinoma patients based upon a 14 gene expression analysis from RNA extracted from FFPE lung adenocarcinoma tissue.

### DETAILED DESCRIPTION OF THE INVENTION

Improved methods for the extraction of total RNA from formalin fixed embedded tissue of sufficient quality and quantity to be reliably measured by Polymerase Chain Reaction technologies are described herein.

Generally, the first step in the methods is the removal of the embedding substance. This is accomplished by dissolving the embedding substance by a technique that may include a solvent that does not destroy RNA. Although a variety of tissue embedding substances are known in the art, most tissue embedding substances are paraffin based. Substances that dissolve paraffin have hydrophobic characteristics and are known in the art. A preferred embodiment is to dissolve the paraffin embedding the tissue sample is to treat the paraffin containing sample with xylene and heat. A preferred heat range is between 60° and 90° C., more preferably between 70° and 85° C., and even more preferably around 80° C. Removal of the xylene from the tissue can be accomplished by washing with another solvent. A preferred solvent for removal of xylene is ethanol.

Embedded tissues may not only trap the desired RNA but may also contain active RNases that destroy the desired product. Therefore, a subsequent step in the method is to free the RNA from protein matrix in which it is trapped or cross-linked and to inactivate RNases (if any). This can be accomplished by treating with a protease, particularly one that is a broad spectrum protease, and more preferably a broad spectrum serine protease, and even more preferably is proteinase K. Thus, in a preferred embodiment the tissue sample is incubated in the presence of an amount of proteinase K effective to accomplish these goals. Preferably the protease is present when the tissue is lysed with lysing solutions. Lysing solutions are known in the art and are commercially available (see, for example, Example 1).

After protease treatment it is desirable to remove the protein and protease from the sample prior to its being treated to remove or degrade the DNA. Methods of separation of protein from nucleic acids are known in the art. A preferred embodiment is to precipitate the protein. Reagents for precipitating proteins without destruction of the desired nucleic acids are known in the art. In a preferred embodiment the protein is precipitated with MPC Protein Precipitation Reagent.

Subsequent to removal of the protein the nucleic acids in solution are precipitated by alcohol. Methods for alcohol precipitation are known in the art. In a preferred embodiment the nucleic acids are precipitated with isopropanol in an amount sufficient to precipitate at least 60%, or at least 70%, or at least 80%, or at least 90%, preferably at least 95%, more preferably at least 96%, and preferably at least 97%, and even more preferably at least 98% of the nucleic acids in solution.

The nucleic acids isolated by precipitation are then treated with a DNAse sufficient to degrade substantially all of the DNA from the preparation. Preferably the DNase is an endonuclease, and more preferably the DNase is DNase 1. This treatment reduces the relative amount of the DNA to RNA to an acceptable level so that it does not cause problems in the subsequent RNA assays. The combination of DNase and RNA purification by silica gel membrane purification yields samples in which substantially all of the DNA is removed, i.e. the ratio of DNA to RNA in the purified sample is usually less than about 0.1%.

After DNase treatment the RNA in the sample is purified using silica gel membrane technology. Preferred silica gel membranes have the RNA, DNA, salt and protein binding characteristics of those in Qiagen RNeasy MinElute® Spin Columns of those in the Qiagen RNeasy MinElute columns Methods of determining RNA, DNA, salt and protein binding to a matrix or membrane are known in the art. In a preferred embodiment Qiagen RNeasy MinElute columns are used. During this part of the method the RNA in the sample is precipitated with alcohol onto the silica gel matrix, washed with buffers to remove the DNA and proteins that are not bound to the silica-gel membrane and to remove salts that are not bound to the silica gel membrane. In a preferred embodiment the buffers are Buffer RW1 to remove the protein and DNA and then with Buffer RPE to remove salts.

RNA purified by the above method is then eluted from the silica gel with RNase free water.

The RNA extracted from the formalin fixed embedded tissue, particularly the FFPE tissue is suitable for use in PCR assays as show below in the Examples. Characteristics of RNAs that are suitable for use in PCR assays and particularly qPCR and ways to measure these characteristics are known in the art. These include sufficient concentration, sufficient purity, and a desirable Ct. In a preferred embodiment the concentration of the RNA and its purity are measured in a Nanodrop Spectrophotometer where the concentration is read in ng/ml, and the purity is determined by the 260/280 ratio and the 260/230 ratio. Desirably, the concentration of the RNA is 50 ng/ml or greater, the 260/280 ration is less than about 1.9, and the 260/230 ratio is less than about 1.0.

Methods of measuring Ct are known in the art. For example, Ct values are the raw untransformed data generated by a qPCR machine. They represent a threshold cycle at which the RNA amplifies. Each qPCR cycle generates twice the material as the previous cycle (log scale 2). For example, a Ct value of 10 means that the cycle amplification threshold for that RNA for the specific primer probe set is 2̂10. A Ct value of 20 means the cycle amplification threshold for that RNA is 2̂20. Cts can be compared. The difference between 2 Cts represents the difference in the amounts of RNA present on a log 2 scale. Usually it is difficult to reliably amplify RNA samples with a Ct of greater than about 30.

All of the above processes are carried out in a manner that introduces or allows little to no RNase activity in the RNA containing samples.

### EXAMPLES

The following examples are offered to illustrate but not to limit the claimed invention

### Materials

Epicentre MasterPure™ RNA Purification Kit MCR85102 that includes: Red Cell Lysis Solution; Tissue and Cell Lysis Solution; MPC Protein Precipitation Reagent; T&C Lysis Solution, TE Buffer; RNase-Free DNase; Protease K; DNase Buffer and RiboGuard™ RNase Inhibitor.

Epicenre MasterPure™ RNA Purification reagents that include: Tissue & Cell Lysis Solution (Manufacturer Part No MTC096H); Proteinase K (Manufacturer Part No MPRK092); and MPC Protein Precipitation Reagent (Manufacturer Part No MMP03750).

Qiagen Rneasy Micro Kit 74004 that includes: RNeasy MinElute® Spin Coumns; Collection Tubes; Buffer RLT; Buffer RW1; Buffer rpe; mASE-Free water; Carrier RNA, polyA; RNase-Free DNase with buffer and RNase-Free water. When used the RLT Buffer contains 10 μl of η-mercaptoethanol per ml of buffer.

RNaseZap® Rnase decontamination solution (Biosystems Part Number 9782)

RNase free water (Fisher Part Number SH30538.03 or equivalent

Molecular grade ethanol (Fisher Part Number PB2818-500 or equivalent)

ACS grade xylene

Beta-mercaptoethanol

Ethanol (standard grade) diluted to the appropriate concentration

### Example 1

### Purification of Total RNA From FFPE Tissue Sections

Paraffin is removed and the sample is lysed by the following procedure. A sample containing three 10 μm sections is placed in a microcentrifuge tube. Xylene is added to the sample, it is heated to 80° C. and vigorously vortexed. The supernatant is removed and the process repeated. 100% ethanol is added to the pellet and the mixture is vortexed and centrifuged; the process is repeated. The resulting pellet is treated with Tissue & Cell Lysis Solution containing Proteinase K, incubated at about 65° C. for about 2 hours, and vortexed.

Protein is removed from the above sample as follows. MPC Protein Precipitation Reagent is added to the chilled sample and it is vortexed and centrifuged at about 13,500 RPM at a temperature of between 2° to 8° C. for about 10 minutes. The supernatant is transferred to a clean tube and nucleic acids are precipitated by the addition of isopropanol (at a ratio of about 5:4 isopropanol to supernatant solution). After mixing the precipitate is collected by centrifugation at about 13,500 RPM at a temperature of between 2° to 8° C. for about 10 minutes. After removal of the supernatant the nucleic acid pellet is rinsed twice with 70% ethanol and the nucleic acids are resuspended in RNase free water.

DNA in the sample is digested by treatment with DNase 1 using RDD buffer containing the DNase. The concentrations and amounts are according to the Manufacturer's direction. (See above for Materials and Manufacturer).

The RNA in the sample is then purified using gel membrane technology as follows. Buffer RLT (300 μl) is added to 100 μl of the DNA digested material, it is mixed, and ethanol (250 μl) is added and the sample is again mixed. The sample is transferred to an RNeasy MinElute spin column in a microfuge tube and the sample is spun at 11,000 rpm for 15 seconds. The spin column is placed in another tube and 700 μl Buffer RW1 is added to the sample in the RNeasy MinElute spin column and the spin column is subjected to centrifugation. This process is repeated. The RNeasy MinElute spin column is placed in a new collection tube and 500 μl of Buffer RPE is added and the column is centrifuged; the process is repeated. 500 μl of 80% ethanol is added to the spin column and it is collected by centrifugation. The RNA in the spin column is then eluted with RNAse free water.

### Example 2

### Comparison of RNA Extraction Methods From FFPE Sections

Aliquots of the same samples were extracted by 3 methods.

Method 1: Silica gel membrane extraction and purification utilized Qiagen RNeasy FFPE Kit Cat. No. 73504 followed by Qiagen RNeasy Micro Kit 74004 and the procedures were according to the Manufacturer's directions.

Method 2: Alcohol based precipitation for both extraction and purification utilized Epicentre MasterPure™ RNA Purification Kit MCR85102 and the procedures were according to the Manufacturer's directions.

Method 3: Combination of alcohol based precipitation and silica-gel technology as described in Example 1 (the PPG Method).

The quantity and quality of the RNA extracted by Methods 1, 2 and 3 were measured by Nanodrop Spectrophotometer readings. These readings are: ng/μl (quantity); 260/280 ratio (purity—higher ratio is better purity); and 260/230 ratio (purity—higher is better). The results are shown in Table 1.

TABLE 1

| Sample | method 1 | | | method 2 | | | method 3 | | |
|---|---|---|---|---|---|---|---|---|---|
| ID | ng/ul | 260/280 | 260/230 | ng/ul | 260/280 | 260/230 | ng/ul | 260/280 | 260/230 |
| 601 | 30.59 | 1.87 | 1.05 | 810.21 | 2.01 | 1.86 | 129.22 | 1.95 | 2.05 |
| 602 | 9.20 | 1.42 | 0.31 | 1238.78 | 2.01 | 1.97 | 138.49 | 1.98 | 1.33 |
| 603 | 14.42 | 1.82 | 0.58 | 1631.87 | 2.01 | 1.79 | 122.51 | 1.99 | 1.57 |
| 604 | 20.51 | 1.54 | 0.29 | 773.55 | 1.96 | 1.84 | 90.80 | 1.97 | 1.58 |
| 605 | 32.29 | 1.53 | 0.28 | 738.73 | 1.95 | 1.63 | 141.01 | 1.98 | 1.58 |
| 606 | 15.81 | 1.62 | 0.48 | 542.95 | 1.94 | 1.58 | 136.53 | 1.99 | 1.84 |
| 607 | 24.32 | 1.49 | 0.20 | 2557.75 | 2.03 | 1.97 | 145.39 | 1.97 | 2.12 |
| 608 | 10.78 | 1.49 | 0.44 | 1604.68 | 1.99 | 1.87 | 140.82 | 1.95 | 1.21 |
| 609 | 15.31 | 1.51 | 0.44 | 1401.01 | 2.01 | 1.82 | 143.36 | 2.02 | 1.31 |
| 610 | 16.28 | 1.51 | 0.36 | 3105.63 | 1.99 | 2.00 | 145.99 | 1.95 | 2.10 |
| 611 | 34.06 | 1.56 | 0.61 | 864.39 | 2.01 | 1.67 | 135.86 | 2.02 | 1.97 |
| 612 | 10.17 | 1.47 | 0.46 | 1017.95 | 2.03 | 1.85 | 140.65 | 1.99 | 1.85 |
| 613 | 17.08 | 1.54 | 0.48 | 2105.38 | 2.04 | 2.05 | 134.60 | 1.95 | 2.18 |
| 614 | 5.78 | 1.45 | 0.37 | 356.54 | 1.42 | 1.14 | 109.70 | 1.91 | 2.16 |
| 615 | 208.33 | 1.88 | 1.34 | 205.62 | 1.79 | 1.25 | 81.54 | 1.98 | 0.95 |
| 616 | 18.79 | 1.48 | 0.42 | 797.88 | 2.01 | 1.72 | 112.05 | 1.98 | 1.80 |
| 617 | 8.77 | 1.54 | 0.33 | 974.63 | 1.98 | 1.82 | 135.89 | 2.01 | 2.06 |
| 618 | 9.28 | 1.59 | 0.43 | 906.20 | 1.91 | 1.78 | 134.30 | 1.97 | 1.99 |
| 619 | 7.50 | 1.48 | 0.09 | 1153.41 | 2.02 | 1.91 | 139.89 | 1.97 | 2.09 |
| 620 | 7.42 | 1.61 | 0.32 | 726.62 | 2.02 | 1.86 | 141.89 | 1.94 | 2.05 |
| 621 | 18.63 | 1.58 | 0.41 | 716.08 | 2.01 | 1.83 | 144.96 | 2.00 | 1.97 |
| 622 | 28.43 | 1.66 | 0.53 | 833.41 | 1.94 | 1.81 | 144.05 | 1.97 | 1.67 |
| 623 | 13.61 | 1.59 | 0.48 | 689.64 | 2.00 | 1.76 | 129.91 | 1.91 | 2.20 |
| 624 | 14.66 | 1.53 | 0.22 | 1236.03 | 2.01 | 1.91 | 45.86 | 1.97 | 0.71 |
| Mean | 24.67 | 1.57 | 0.46 | 1124.54 | 1.96 | 1.78 | 127.72 | 1.97 | 1.76 |
| STDEV | 39.96 | 0.12 | 0.26 | 676.62 | 0.13 | 0.21 | 24.26 | 0.03 | 0.41 |

As can be seen above, methods 2 and 3 are superior to method 1. It is very difficult to reliably amplify RNA if the concentration is less than 50 ng/ml, the 260/280 ratio is less than 1.9, and the 260/230 ratio is less than 1.0. The number of samples meeting these criteria for each method is shown in Table 2.

TABLE 2

| | No. successful extractions | Percentage |
|---|---|---|
| method 1 | 0 | 0% |
| method 2 | 22 | 91.60% |
| method 3 | 23 | 95.80% |

### Example 3

### Comparison of Amplification of RNA From Different Extraction Methods in Quantitative PCR

Methods 1, 2 and 3 are as described in Example 2. RNA was extracted from 24 samples using each technique. RNA quantity was determined by measuring the relative expression of 18S, a housekeeping gene, by quantitative PCR (qPCR). Each 1 unit decrease in 18S Raw Ct value indicates a 2-fold higher quantity of extracted RNA as measured by qPCR.

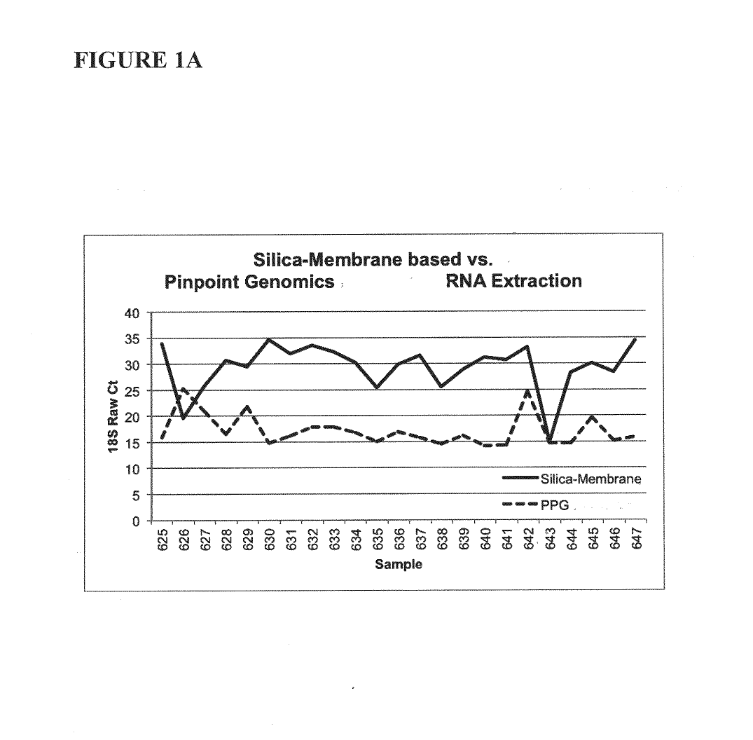

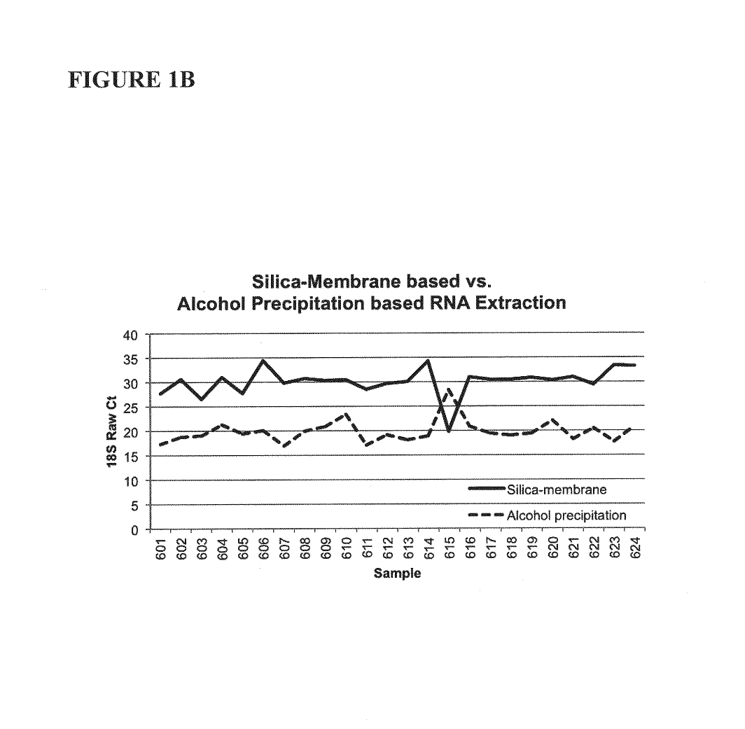

The comparison of the Ct values of Method 1 vs. Method 2 is shown in FIG. 1A, and of Method 2 vs. Method 3 is shown in FIG. 1B.

A summary of the mean Raw Ct value and standard deviations of the samples extracted using the different extraction methods as measured by qPCR is shown in Table 3.

TABLE 3

| Method | 18S Mean Raw Ct | SD |
|---|---|---|
| 1. Silica-Membrane based | 29.733 | 3.856 |
| 2. Alcohol-Precipitation based | 19.882 | 2.413 |
| 3. PPG | 17.211 | 3.196 |

### Example 4

### Comparison of PPG Method Extracted RNA From PPFE to RNA Extracted From Frozen Tissue

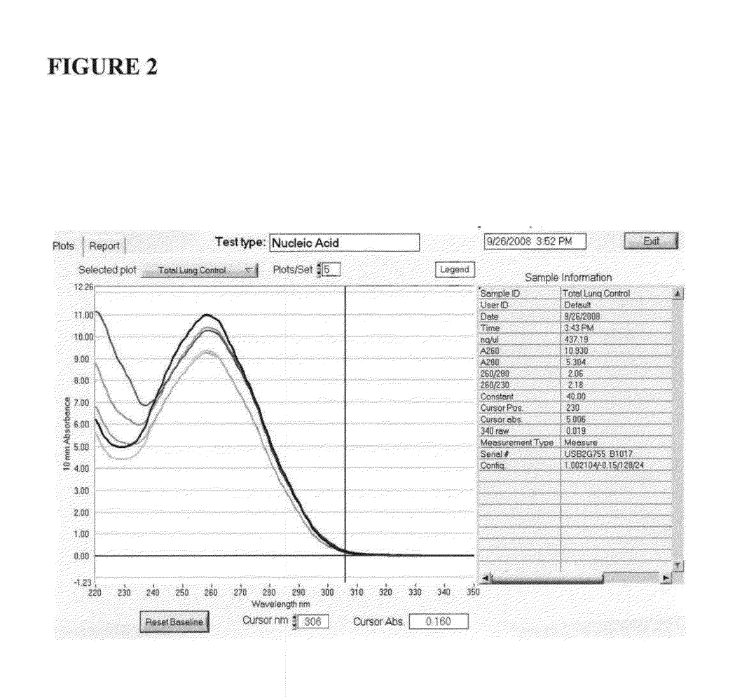

RNA was extracted from 4 PPFE samples by the PPG method (see Example 1) and was compared by Nanodrop spectrophotometry to commercially prepared RNA extracted from frozen tissues. The results demonstrated that the quantity and quality of the samples were very similar. The results are shown in FIG. 2. The black line trace is that of the RNA extracted from frozen tissues.

### Example 5

### Use of RNA Extracted By PPG Method From PPFE Lung Adenocarcinoma Tissue in Prognosis Based Upon a 14 Gene Assay

RNA was extracted from FFPE sections essentially as described in Example 1. RNA was extracted from six 10-micron FFPE sections after proteinase K treatment using alcohol-based nucleic acid precipitation (MasterPure RNA Purification Kit, Epicentre, Madison, Wis.). RNA extracts were DNase-treated and purified using silica-gel-membrane spin columns (Qiagen, Valncia, Calif.) and RNA quantity and quality was measured using a Nanodrop spectrophotometer (Thermo Scientific, Wilmington, Del.). Extracted RNA underwent reverse transcription using gene-specific priming (iScript cDNA Synthesis Kit, BioRad Laboratories, Hercules, Calif.) followed by cDNA preamplification (TaqMan PreAmp Master Mix, Applied Biosystems, Carlsbad, Calif.). TaqMan quantitiative PCR assays (BioSearch Technologies, Novato, Calif.) custom-designed for use on RNA extracted from FFPE tissues were used to quantify RNA expression using FAST chemistry on a 7900 HT Fast Real-Time PCR System (Applied Biosystems, Carlsbad, Calif.). FFPE-specific TaqMan quantitive PCR assays were designed to target 65-85 base pair amplicons that crossed exon-exon boundaries, avoiding template structures and cross-homologies (Beacon Designer 7.0, Premier Biosoft, Palo Alto, Calif.). All primer sequences underwent a BLAST search against the human genome (NCBI ref_assembly 37.1) to ensure target specificity. Synthesized primers were tested for optimal primer concentrations and single product dissociation. All RNA expression measurements were normalized to RNA extracted from pooled frozen normal lung samples (Clontech Laboratories, Mountain View, Calif.) and the relative expression for each target gene was calculated using the comparative CT method.

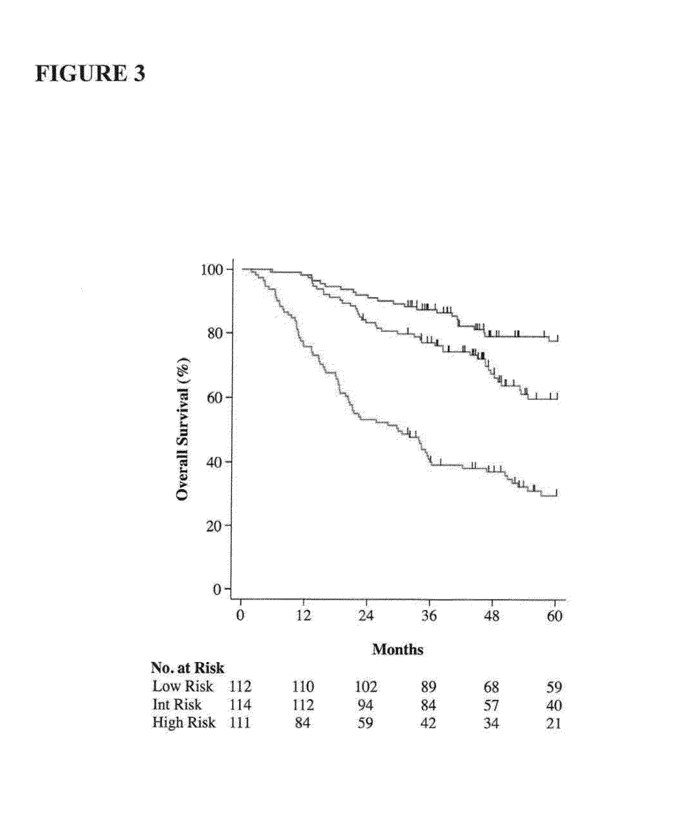

The Prognostic algorithm development and statistical analysis were as follows. Eleven cancer-related target genes (BAG1, BRCA1, CDC6, CDK2AP1, ERBB3, FUT3, IL11, LCK, RND3, SH3BGR, WNT3A) and three candidate reference genes (ESD, TBP, and YAP1) were evaluated in a cohort. The eleven target genes were selected using L1-penalized Cox proportional hazards modeling from a large pool originally consisting of over 200 cancer-related genes identified from previously published microarray and PCR-based studies of prognosis in early stage lung cancer part of a prior study. A pilot study on fifty FFPE samples revealed ESD, TBP and YAP1 to be the most stable reference genes (stability analyzed using geNorm and NormFinder). In order to minimize overfitting of the training dataset, L2-penalized Cox proportional hazards modeling was the primary analytical tool used to develop the prognostic algorithm. The amount of L2-penalty applied was determined using 10-fold cross-validation29. A continuous risk score was generated for each subject based on model coefficients; resultant predicted risk scores were scaled to a range between 1-100, then divided at the 33^{rd} and 67^{th} percentiles to generate low, intermediate, and high-risk groups. The data obtained from this were used to determine prognosis as shown in FIG. 3.

Samples for above were also examined using the silica-gel method (Method 1 as described in Example 2). The Ct values for the average housekeeping gene ((ESD-1, TBP-2, YAP1-1) were compared utilizing Method 1 with those obtained utilizing the PPG Method (Method 3). It is very difficult to reliably amplify samples with an average housekeeping gene Ct greater than 30. The results showed that using Method 1 410/479 samples (85.6%) met a criteria of a Ct less than 30, while 477/484 (98.6%) of the Method 3 samples met this criteria.

What is claimed is:

1. A method for extracting RNA from a sample of formalin fixed embedded tissue, the method comprising: (a) removing the embedding material; (b) incubating the tissue with an effective amount of a protease to dissolve protein cross-links and inactivate RNase (if any); (c) removing protein from nucleic acids; (d) precipitating nucleic acids; (e) removing DNA from the nucleic acids; and (e) purifying the RNA by binding to and eluting from a silica gel membrane, wherein the extracted RNA from the sample is suitable for use in a PCR assay.

2. The method of claim 1 wherein the sample is embedded in a paraffin containing material.

3. The method of claim 2 wherein removing the embedding material is by solvents.

4. The method of claim 3 wherein a solvent is xylene.

5. The method of claim 2 wherein the protease has Proteinase K type of activity.

6. The method of claim 5 wherein protease is Proteinase K.

7. The method of claim 2 wherein the nucleic acids are precipitated with an effective amount of an alcohol.

8. The method of claim 7 wherein the alcohol is isopropanol.

9. The method of claim 2 wherein DNA removal comprises digestion with an effective amount of a DNase.

10. The method of claim 9 wherein the DNase is a broad spectrum endonuclease.

11. The method of claim 10 wherein the DNase is DNase 1.

12. The method of claim 2 wherein the silica gel membrane has RNA, DNA, salt and protein binding characteristics of those in Qiagen RNeasy MinElute® Spin Columns

13. The method of claim 12 wherein the silica gel membrane is in Qiagen RNeasy MinElute® Spin Column
